(19) United States
(12) Patent Application Publication
(10) Pub. No.: US 2021/0000135 A1
(43) Pub. Date: Jan. 7, 2021

(54) PROTEIN COMPOSITION

(71) Applicant: MEIJI CO., LTD., Tokyo (JP)

(72) Inventors: Nobuko JINNO, Tokyo (JP); Yoshio TOYAMA, Tokyo (JP)

(73) Assignee: MEIJI CO., LTD., Tokyo (JP)

(21) Appl. No.: 16/976,836

(22) PCT Filed: Feb. 28, 2019
(86) PCT No.: PCT/JP2019/007797

§ 371 Date: Aug. 31, 2020

(30) Foreign Application Priority Data
Mar. 1, 2018 (JP) 2018-036943

(51) Int. Cl. A23J 3/10; A23J 1/20; A23J 1/22; A23L 29/269; A23L 29/212; A23L 29/30; G01N 33/04; A23P 10/20

(52) U.S. Cl. CPC A23J 3/10; A23J 1/202; A23J 1/205; A23J 1/22; A23P 10/20; A23L 29/212; A23L 29/30; G01N 33/04; A23L 29/273

(57) ABSTRACT

Provided are a protein powder capable of increasing the amount of protein ingested, a protein-containing composition, a method for producing a food, a method for producing a protein powder, a method for producing a protein-containing composition, a method for evaluating a protein powder, and a method for evaluating a protein-containing composition. A water droplet is adhered to the surface of the protein powder which was in a leveled state to measure a contact angle. The contact angle is measured from the time point when the water droplet contacts with the protein powder, and it is determined whether or not the contact angle is 90° or less within 60 seconds. The protein powder having a contact angle of 90° or less within 60 seconds is evaluated as a protein powder that is easy to eat without water.

### TECHNICAL FIELD

The present invention relates to a protein powder, a protein-containing composition, a method for producing a food, a method for producing a protein powder, a method for producing a protein-containing composition, a method for evaluating a protein powder, and a method for evaluating a protein-containing composition.

### BACKGROUND ART

Examples of the food for eating the protein include a protein powder dissolved in water, a liquid food, a protein-containing beverage, a protein-containing jelly, and the like. The powder may generally be granulated for ease of eating. Patent Literature 1 discloses a powdered nutritional composition that can be ingested as a powder. Further, the powdered nutritional composition of Patent Literature 1 contains a fat or oil containing docosahexaenoic acid, a calcium-containing component, and at least one or more of palatinose and a sugar alcohol, and can be ingested as a powder.

### CITATION LIST

### Patent Literature

[Patent Literature 1] JP-A-8-56612

### SUMMARY OF INVENTION

### Technical Problem

When the protein is ingested, the protein powder needs to be dissolved in water, and the protein powder cannot be ingested in a place where there is no water such as outside or in an environment where a hygienic water cannot be obtained, and it takes time to dissolve the protein even when the appropriate water is obtained. In addition, when the protein powder is dissolved in a liquid, the volume to be ingested increases, and it is difficult for a person suffering from anorexia to ingest the protein powder. Liquid foods, protein-containing beverages, protein-containing jellies, and the like are easy to eat, but contain a large amount of lipids, carbohydrates, and the like, and the amount of protein that can be ingested is small relative to the amount that enters the mouth. For this reason, when a person with a reduced amount of food or a person with calorie restriction tries to ingest a necessary amount of protein, there arises a problem that the person cannot drink or eat the protein. In addition, the granulated powder requires a granulation step, which increases the production cost. For these reasons, there is a demand for an easy-to-eat food capable of increasing the amount of protein ingested while reducing the amount of protein entering the mouth.

The present invention has been made in view of the above circumstances, and an object of the present invention is to provide a protein powder, a protein-containing composition, a method for producing a food, a method for producing a protein powder, a method for producing a protein-containing composition, a method for evaluating a protein powder, and a method for evaluating a protein-containing composition, which can increase the amount of protein ingested while reducing the amount of protein entering the mouth.

### Solution to Problem

The present inventors have found that a protein powder having a small contact angle with water has properties of being easily dispersed in saliva, hardly forming lumps, hardly removing water in the oral cavity, and hardly adhering to the mouth when ingested, and that such a protein powder is easily eaten because the time required for swallowing all the protein powder is shortened even when ingested without water. In addition, the present inventors have found that a protein-containing composition obtained by adding a saccharide to a protein powder having a small contact angle with water tends to require a shorter time for swallowing all the protein-containing composition than the protein powder alone. The present invention is based on these findings.

A protein powder according to the present invention has a contact angle with water of 90° or less within 60 seconds from the time point of contact with water.

A protein-containing composition according to the present invention is a mixture of the protein powder and a saccharide.

A method for producing a food according to the present invention, includes a determination step of measuring a contact angle of a protein powder with water to determine whether or not a contact angle condition is satisfied on the condition that the measured contact angle of the protein powder is 90° or less within 60 seconds from the time point of contact with water; and a food preparation step of processing the protein powder satisfying the contact angle condition to prepare a food.

In the method for producing a food according to the present invention, the protein powder having a contact angle with water of 90° or less within 60 seconds from the time point of contact with water is used in the process for producing the food containing the protein powder.

A method for producing a protein powder according to the present invention, includes a determination step of producing a protein powder for measurement from a raw material and measuring a contact angle of the protein powder for measurement with water, to determine whether or not a contact angle condition is satisfied on the condition that the measured contact angle of the protein powder is 90° or less within 60 seconds from the time point of contact with water; and a production step of producing a protein powder from the raw material of the protein powder for measurement determined to satisfy the contact angle condition.

A method for producing a protein-containing composition according to the present invention, includes a determination step of producing a protein powder for measurement from a raw material and measuring a contact angle of the protein powder for measurement with water, to determine whether or not a contact angle condition is satisfied on the condition that the measured contact angle of the protein powder is 90° or less within 60 seconds from the time point of contact with water; a production step of producing a protein powder from the raw material of the protein powder for measurement determined to satisfy the contact angle condition; and a mixing step of mixing the produced protein powder with a saccharide.

A method for evaluating a protein powder according to the present invention, includes a powder preparing step of putting a protein powder into a container and flattening the surface of the protein powder by leveling; an adhesion step of adhering a water droplet to the surface of the protein powder; a measurement step of measuring a contact angle of the water droplet on the surface of the protein powder; and an evaluation step of evaluating the ease of eating the protein powder based on whether or not the contact angle is 90° or less within 60 seconds from the time point when the water droplet contacts the surface of the protein powder.

A method for evaluating a protein-containing composition according to the present invention, includes a protein-containing composition preparing step of putting a protein-containing composition obtained by mixing a protein powder and a saccharide into a container and flattening the surface of the protein-containing composition by leveling; an adhesion step of adhering a water droplet to the surface of the protein-containing composition; a measurement step of measuring a contact angle of the water droplet on the surface of the protein-containing composition; and an evaluation step of evaluating the ease of eating the protein-containing composition based on whether or not the contact angle is 90° or less within 60 seconds from the time point when the water droplet contacts the surface of the protein-containing composition.

### Advantageous Effects of Invention

According to the present invention, since the contact angle of the protein powder with water is 90° or less within 60 seconds from the time point of contact with water, an easy-to-eat protein powder or food can be provided, so that the amount of protein ingested can be increased while reducing the amount of protein entering the mouth.

According to the protein-containing composition of the present invention, the protein powder can be made easier to eat by mixing the saccharide, and the production process for making it easier to eat can be simplified.

According to the method for evaluating a protein powder of the present invention, the contact angle is measured with a water droplet adhered to the surface of the protein powder in a leveled state, and the ease of eating is evaluated based on whether or not the contact angle becomes 90° or less within 60 seconds from the time point of contact with the water droplet. Therefore, it is possible to easily evaluate the ease of eating the protein powder without water.

### BRIEF DESCRIPTION OF DRAWINGS

FIG. 1 is an explanatory diagram showing protein powders according to an example of an embodiment of the present invention.

FIG. 2 is an explanatory diagram showing a procedure of a powder preparing step for measuring a contact angle of protein powders.

FIG. 3 is an explanatory diagram showing states of water droplets and protein powders in an adhesion step and a measurement step.

FIG. 4 is a process chart showing an example of a production procedure of protein powders.

FIG. 5 is an explanatory diagram showing an example of a protein-containing composition.

### DESCRIPTION OF EMBODIMENTS

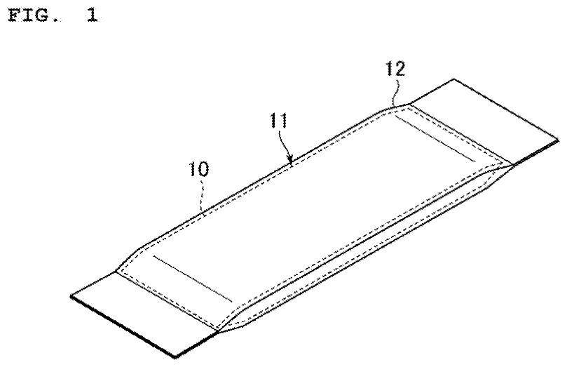

In FIG. 1, a protein powder 10 according to an embodiment of the present invention is provided in a sealed pouch 12 as a food 11 for each dose or each predetermined amount. The protein powder 10 is put into the oral cavity of the person who ingests it from the pouch 12 at the time of ingestion. The protein powder 10 satisfies the contact angle condition described later, and is a powder to be eaten without water, which is easily eaten without a liquid such as water. The form of providing the protein powder 10 is not particularly limited. For example, the protein powder 10 may be provided in a large-capacity container, and when ingested, a desired amount of the protein powder 10 may be scooped out with a spoon or the like from a container such as a large bag, a can of powdered milk, or a plastic case of baby powder.

The protein used as the protein powder 10 is not particularly limited, and examples thereof include milk protein-derived proteins, egg protein-derived proteins, soybean protein-derived proteins, and wheat protein-derived proteins. In particular, those derived from milk proteins can be preferably used. The milk protein-derived protein is not particularly limited, and examples thereof include those derived from Micellar Casein Concentrate (MCC), Whey Protein Concentrate (WPC), Whey Protein Isolate (WPI), whey powder, whey protein hydrolysate, Milk Protein Concentrate (MPC), Milk Protein Isolate (MPI), milk protein enzymatic degradation products, milk protein fractions (e.g., α-casein, β-casein, κ-casein, β-lactoglobulin, α-lactealbumin, lactoferrin, etc.), whole powdered milk, and powdered skim milk. Preferred examples include those derived from Micellar Casein Concentrate, Whey Protein Concentrate, Whey Protein Isolate, whey protein hydrolysate, Milk Protein Concentrate, Milk Protein Isolate, and milk protein enzymatic degradation products. More preferred are those derived from Micellar Casein Concentrate, Whey Protein Concentrate, and Milk Protein Concentrate. Further, two or more kinds of the above-mentioned milk proteins may be combined. The protein powder 10 contains a component corresponding to a material from which the powder is derived, and may not contain only protein as a component.

For example, the protein powder 10 composed of Micellar Casein Concentrate is produced by sterilizing raw milk, skim milk, skimmed condensed milk, or the like at a high temperature for a short time, followed by membrane separation, concentrating, and spray-drying. The protein powder 10 composed of Whey Protein Concentrate is produced by membrane-concentrating and spray-drying whey, which is a by-product of production of cheese, casein, or the like. The protein powder 10 composed of Milk Protein Concentrate is produced by centrifuging raw milk, followed by sterilizing, ultrafiltering, concentrating, and spray-drying.

The contact angle condition is that a contact angle equal to or smaller than a predetermined contact angle is exhibited within a predetermined time from the time point when water contacts with the protein powder 10. The predetermined time is 60 seconds. The predetermined contact angle is 90°. The protein powder 10 exhibiting a contact angle equal to or smaller than a predetermined contact angle within a predetermined time has characteristics such that it is easily dispersed in saliva (it is difficult to form lumps), it is difficult to remove water in the oral cavity when ingested, and it is difficult to adhere to the inside of the mouth, and it is easy to eat without water. As for the ease of eating, the length of the time (which may be chewed) required from the time when the protein powder 10 is put into the oral cavity to the time when all of the protein powder 10 can be swallowed without water is used as an index, and the shorter the time until the protein powder 10 can be swallowed, the easier the protein powder 10 can be eaten. The smaller the contact angle of the protein powder 10 within the predetermined time, the shorter the time required for the protein powder 10 to be swallowed, and the smaller the contact angle, the easier the protein powder 10 can be eaten. The predetermined contact angle is preferably 85°, more preferably 80°, even more preferably 75°, and particularly preferably 70°, and the smaller the predetermined contact angle, the higher the certainty of the ease of eating the protein powder 10.

In the case of the protein powder 10 having a high Ca (calcium) concentration, the contact angle with water at the time point when a predetermined time (for example, 60 seconds) has elapsed tends to be small. Therefore, the protein derived from milk protein and having a high Ca concentration is advantageous in producing the protein powder 10 that is easy to eat. In addition, the inventors have found that the higher the Ca concentration of the protein powder 10 composed of milk protein concentrate is, the shorter the time required for the protein powder to be eaten and swallowed is, and a preferable effect is obtained.

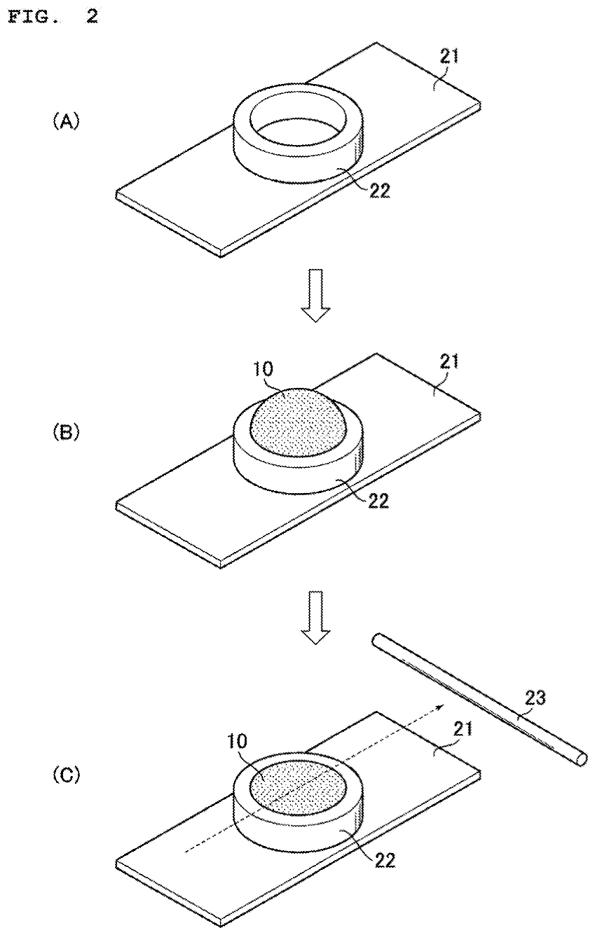

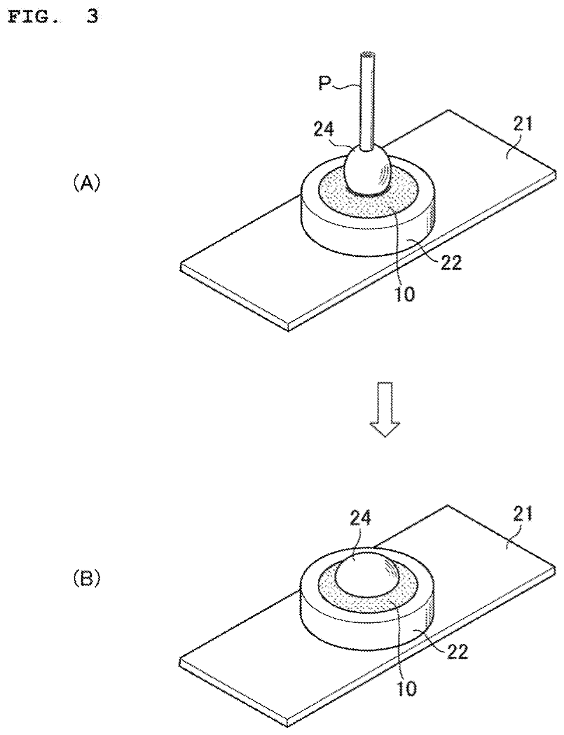

Hereinafter, the measurement of the contact angle of the protein powder 10 with water will be described with reference to FIG. 2 and FIG. 3. In this example, the contact angle of the protein powder 10 with water is measured by a powder preparing step, an adhesion step, and a measurement step. Further, in this example, as shown in FIG. 2(A), a container in which a ring member 22 is placed on a slide glass 21 is used. The ring member 22 has a constant edge height so that the surface of the protein powder 10 becomes a horizontal flat surface when leveling of the protein powder 10 put into the ring member 22 is performed. Note that the container is not limited to the one having the above-described configuration, and any container may be used as long as the surface of the protein powder 10 can be made flat when being leveled off.

In the powder preparing step, as shown in FIG. 2(B), the protein powder 10 is put into the ring member 22. Thereafter, as shown in FIG. 2(C), a leveling member, in this example, a glass rod 23 is used to level off the protein powder 10. That is, the protein powder 10 above the edge of the ring member 22 is removed to flatten the surface of the protein powder 10.

In the adhesion step after the powder preparing step, as shown in FIG. 3(A), a water droplet 24 is dropped from a pipe P onto the surface of the leveled protein powder 10, and is adhered to the surface of the protein powder 10 as shown in FIG. 3(B). Thereafter, in the measurement step, the contact angle of the water droplet 24 on the surface of the protein powder 10 is measured. For example, after the powder preparing step, the slide glass 21 and the ring member 22 as well as the protein powder 10 in the leveled state are set in a contact angle meter, and the adhesion step and the measurement step are performed by the contact angle meter.

In the case of determining whether or not the protein powder 10 satisfies the above-described contact angle condition, the contact angle is measured as described above, and it is checked whether or not the contact angle measured within a predetermined time (for example, within 60 seconds) after the water droplet 24 comes into contact with the surface of the protein powder 10 is equal to or less than a predetermined angle (for example, 90°). It is determined that the contact angle condition is satisfied when the contact angle becomes equal to or less than the predetermined angle within the predetermined time after the water droplet 24 comes into contact with the surface of the protein powder 10, and it is determined that the contact angle condition is not satisfied when the contact angle does not become equal to or less than the predetermined angle within the predetermined time. The protein powder 10 determined to satisfy the contact angle condition as described above can be evaluated as easy to eat even without water. It is also possible to check whether or not the contact angle at the time point when a predetermined time has elapsed after the water droplet contacts the protein powder 10 is equal to or less than a predetermined angle. In this case, it is possible that the water droplet on the protein powder 10 disappears at the time point when the water droplet permeates into the protein powder and the predetermined time has elapsed. In the case of the water droplet disappears, it is determined that the contact angle condition is satisfied.

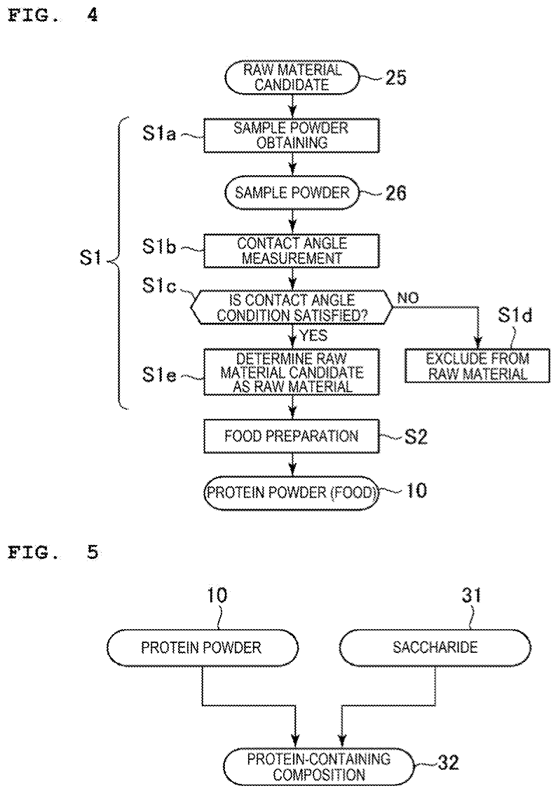

FIG. 4 shows an example of the procedure of the production process of the protein powder 10 to be the food 11. First, in a determination step S1, it is determined whether or not the contact angle condition is satisfied by using a sample powder (protein powder for measurement) 26 produced from a raw material candidate 25 of the protein powder 10.

For example, the sample powder 26 is obtained by being produced from a part of the raw material candidate 25 under the same conditions as in the case of producing the protein powder 10 (step S1a). The obtained sample powder 26 is measured for contact angle with water by the above-described procedure (step S1b), and it is determined whether or not the contact angle condition is satisfied (step S1c). When the contact angle condition is not satisfied in this determination, the raw material candidate 25 is excluded from the raw material of the protein powder 10 to be the food 11 (S1d). On the other hand, when the contact angle condition is satisfied, the raw material candidate 25 is used as the raw material of the protein powder 10 to be the food 11 (S1e). In a food preparation step S2, the protein powder 10 to be the food 11 is produced from the raw material. The protein powder 10 thus produced is weighed in a single dose or in a predetermined amount and accommodated in the pouch 12, and the pouch 12 is sealed.

A part of the protein powder 10 produced from the total amount of the raw material candidate 25 may be obtained as the sample powder 26, and it may be determined whether or not the sample powder 26 satisfies the contact angle condition. In this case, when the contact angle condition is not satisfied and the raw material candidate 25 is excluded from the raw material, the protein powder 10 produced from the raw material candidate 25 is excluded from being provided as the food 11 that is easy to eat. When the raw material candidate 25 is used as the raw material, the protein powder 10 produced from the raw material candidate 25 is used as the food 11 that is easy to eat.

Since the protein powder 10 satisfying the contact angle condition as described above is easy to eat even by itself alone, the protein can be efficiently ingested. That is, even if the amount of the protein powder 10 ingested is small, a large amount of protein ingested can be obtained. Thus, for example, even in a situation where there is no water such as at a place where the person is out, the person can easily ingest a required amount of protein. In addition, since the protein powder 10 satisfying the contact angle condition is easy to eat even without water, it is advantageous because the amount of water ingested does not increase, and it is suitable as a food for supplying protein to such as malnourished people in an area where water is contaminated.

The protein powder 10 satisfying the above-described contact angle condition can be provided as a protein-containing composition together with other powders, and can be more easily eaten without a liquid such as water than the protein powder 10 alone. For example, FIG. 5 shows an example of a protein-containing composition 32 in which a saccharide 31 is added to the protein powder 10 satisfying the above-described contact angle condition. By eating the protein powder 10 together with the saccharide 31, the time required for swallowing can be made shorter than that of the protein powder 10 alone, and the food can be easily eaten without water. The ratio of the protein powder 10 to the saccharide 31 is not particularly limited, but may be, for example, 1:1 to 9:1 (mass ratio). Such a protein-containing composition 32 is produced by merely mixing the protein powder 10 and the saccharide 31 at a predetermined ratio, and is easier to produce than in the case of granulation.

As the saccharide 31, a saccharide generally used for food can be used. Specific examples of the saccharide 31 include monosaccharides such as fructose, glucose, tagatose, and arabinose; disaccharides such as lactose, trehalose, maltose, and sucrose; polysaccharides such as dextrin, cyclodextrin, and starch; crystalline saccharides including polysaccharides such as powdered starch syrup; oligosaccharides such as maltooligosaccharide, galactooligosaccharide, fructooligosaccharide, and lactosucrose; non-crystalline saccharides such as starch syrup and isomerized sugar syrup (for example, corn syrup and high fructose corn syrup); sugar alcohols such as xylitol, sorbitol, glycerin, and erythritol; and rare sugar such as psicose.

A salivation promoting substance may be added to the protein powder 10 to form a protein-containing composition that can be easily eaten without water. By adding the salivation promoting substance, the amount of saliva is increased, and the food can be more easily eaten without water. Examples of the salivation promoting substance include organic acids and polyglutamic acid.

The saccharide 31 and the salivation promoting substance to be mixed with the protein powder 10 may be in any form such as liquid, powder or granular form. The form of the food containing the protein powder 10 or the protein-containing composition is not particularly limited, but it is needless to say that the food or the protein-containing composition is preferably in a form that is ultimately easy to eat.

### Examples

Table 1 shows the results of Experiments 1 to 7 in which the relationship between the contact angle of various protein powders and the oral processing time, which is an index of ease of eating, was examined. In Experiments 1 to 4, protein powders of micellar casein concentrates (MCC1 to MCC4) having different components were examined, in Experiment 5, protein powders of sodium caseinate (NaCN) were examined, and in Experiments 6 and 7, protein powders of whey protein concentrates (WPC1 and 2) having different components were examined. Sodium caseinate is produced by neutralizing milk protein casein, which is obtained by acid treatment of raw milk, skim milk, skimmed condensed milk, or milk products, with an alkali to form a sodium salt, and drying the sodium salt.

In Experiments 1 to 7, the contact angle of the protein powder with water was measured by the same procedure as the above-described measurement procedure, and the contact angle at the time point when 60 seconds had elapsed after the water contacted the protein powder was defined as the contact angle θa. When water droplets disappeared from the surface of the protein powder before the lapse of 60 seconds, the minimum measurable contact angle was defined as the contact angle θa. In addition, the contact angle at the time point when 300 seconds had elapsed after the water contacted the protein powder was measured in the same manner as the contact angle θa and defined as the contact angle θb. In Experiments 1 to 7, measurements were performed 5 times using a new protein powder for each measurement, and the averages thereof are shown in Table 1 as contact angles θa and θb. In Experiments 1 to 4, the contact angle θb was not measured because water droplets disappeared earlier than the elapse of 60 seconds.

In the measurement of the contact angles, the ring member 22 made of a plastic ring having an inner diameter of 13.68 mm and a height of 1.98 mm was placed on the slide glass 21, and the protein powder was put into the ring member 22 and leveled with the glass rod 23 to flatten the surface of the protein powder. The contact angle was measured using a contact angle meter (DM-501: manufactured by Kyowa Interface Science Co., Ltd.) by dropping 10 μl of water (distilled water) onto the protein powder, starting measurement of the contact angle at the time point when 1 second had elapsed after the droplet was dropped, and measuring the contact angle at intervals of 1 second for 1 minute.

The oral processing time is an index of ease of eating and is a time measured as described below. In Experiments 1 to 7, measurement was performed 3 times each, and the average thereof is shown in Table 1 as the oral processing time Ta. The number of subjects is 2, and the mouth is rinsed after each measurement, followed by a rest of 1 minute before the next measurement.

1. Weigh the protein powder with a spatula to 0.3 g.

2. The weighed protein powder (0.3 g) is placed on the center of a tongue of a subject, and the upper jaw is brought into contact with the tongue and allowed to stand.

3. After a lapse of 10 seconds from the standing, the subject is made to eat the protein powder immediately and swallow all of the protein powder. At this time, chewing may be performed.

4. A period of time from when 10 seconds have elapsed from the standing to when the subject swallows all of the protein powder is measured as the oral processing time.

Table 1 also shows the Ca concentration of each protein powder. The Ca concentrations of the protein powders used in Experiments 1 to 7 were measured by ICP emission spectrometry.

TABLE 1

| | Compo- | θa | θb | Ta | Ca concentration |
|---|---|---|---|---|---|
| | nent | (°) | (°) | (sec) | (mg/100 g) |
| Experiment 1 | MCC1 | 6 | — | 31.0 | 2191 |
| Experiment 2 | MCC2 | 10 | — | 31.7 | 2420 |
| Experiment 3 | MCC3 | 19 | — | 33.8 | 2164 |
| Experiment 4 | MCC4 | 34 | — | 34.3 | 2213 |
| Experiment 5 | NaCN | 97 | 94 | 120 or longer | 40 |
| Experiment 6 | WPC1 | 89 | 75 | 59.2 | 460 |
| Experiment 7 | WPC2 | 78 | 38 | 64.4 | 400 |

It can be seen from Table 1 above that the smaller the contact angle θa is, the shorter the oral processing time Ta tends to be. If the contact angle θa is 90° or less, that is, if the protein powder shows 90° or less within 60 seconds from the time point of contact with water, the oral processing time Ta does not become excessively long (70 seconds or less), and it is understood that the protein powder is easy to eat.

When the protein powders of Experiments 1 to 4, which are powders of micellar casein concentrate using casein as a raw material, are compared with the protein powder of sodium caseinate of Experiment 5, it can be seen that the oral processing time Ta of the protein powders of Experiments 1 to 4 is extremely short (40 seconds or less), and the protein powders are easy to eat without water. In addition, it can be seen that the protein powders of Experiments 1 to 4 have a shorter oral processing time Ta than the protein powders of Experiments 6 and 7 and are easy to eat without water. These results indicate that the protein powders of micellar casein concentrates used in Experiments 1 to 4 are easier to eat without water than the protein powders derived from other milk proteins.

Table 2 shows the results of Experiments 8 to 11 in which the relationship between the Ca concentration, the oral processing time, and the contact angle was examined using four types of milk protein concentrate powders having different Ca concentrations. The Ca concentrations of the protein powders of Experiments 8, 9, and 11 were measured by ICP emission spectrometry, and the Ca concentration of the protein powder of Experiment 10 was measured by spectrometry. The contact angles θa and θb and the oral processing time Ta were measured in the same manner as in Table 1. In Experiment 8, the contact angle θb was not measured because the water droplet disappeared earlier than 60 seconds.

TABLE 2

| | Compo- | Ca concentration | θa | θb | Ta |
|---|---|---|---|---|---|
| | nent | (mg/100 g) | (°) | (°) | (sec) |
| Experiment 8 | MPC1 | 2040 | 16 | — | 32.0 |
| Experiment 9 | MPC2 | 1440 | 98 | 47 | 44.5 |
| Experiment 10 | MPC3 | 970 | 92 | 83 | 60.8 |
| Experiment 11 | MPC4 | 300 | 98 | 90 | 120 or longer |

It can be seen from Table 2 above that the milk protein concentrate powders of Experiments 8 to 11 each have a shorter oral processing time Ta as the Ca concentration is higher, and are easier to eat without water. In the protein powders of Experiments 9 to 11, the difference in the contact angle θa at the time point when 60 seconds had elapsed was small, but the difference in the contact angle θb at the time point when 300 seconds had elapsed was significant, and the contact angle θb decreased as the Ca concentration increased.

Table 3 shows the results of Experiments 12 to 14 in which the oral processing time was measured for the protein-containing composition. The protein-containing composition of Experiment 12 was prepared by mixing a protein powder of micellar casein concentrate and corn syrup at a mass ratio of 1:1. The protein powder of the micellar casein concentrate used in Experiment 12 is the same as that of Experiment 1. The protein-containing compositions of Experiments 13 and 14 were prepared respectively by mixing a protein powder of whey protein concentrate and corn syrup at a mass ratio of 1:1. The protein powder of the whey protein concentrate used in Experiment 13 is the same as that of Experiment 6 and the protein powder of the whey protein concentrate of Experiment 14 is the same as that of Experiment 7. Table 3 also shows the oral processing time Ta of Experiments 1, 6, and 7 corresponding to the protein powder contained in the protein-containing composition.

The oral processing time Tb was measured in the same manner as the above-described oral processing time Ta, except that the amount of the protein-containing composition placed on the tongue was set to 0.75 g and the number of subjects was 5, and the oral processing time Tb was measured one time for each protein-containing composition for each subject. Table 3 shows the average of the intraoral processing time Tb of 5 subjects. After the measurement of the oral processing time Tb, a change in physical properties (whether or not the composition adheres to teeth) during the oral processing for swallowing the protein-containing composition was asked, and sensory evaluation thereof was performed.

TABLE 3

| | | | Number of | | | |
|---|---|---|---|---|---|---|
| | | | subjects | Reference | | |
| | Compo- | Tb | adhering | | Compo- | Ta |
| | nent | (sec) | to teeth | | nent | (sec) |
| Experi- | MCC1 + corn | 28.0 | 1/5 | Experi- | MCC1 | 31.0 |
| ment 12 | syrup | | | ment 1 | | |
| Experi- | WPC1 + corn | 54.2 | 5/5 | Experi- | WPC1 | 59.2 |
| ment 13 | syrup | | | ment 6 | | |
| Experi- | WPC2 + corn | 23.8 | 0/5 | Experi- | WPC2 | 64.4 |
| ment 14 | syrup | | | ment 7 | | |

As can be seen from Table 3, in all of Experiments 12 to 14, the oral processing time Tb was shorter than the oral processing time Ta of the protein powder alone, although the amount of the powder placed on the tongue was larger than that in the case where the oral processing time Ta of the protein powder alone was measured. From these results, it can be seen that the protein-containing composition obtained by adding corn syrup to the protein powder of the protein concentrate is effective in enhancing the ease of eating. The oral processing time Tb of Experiment 14 is significantly shorter than the oral processing time Ta. On the other hand, the oral processing time Tb of Experiment 13 was not significantly different from the oral processing time Ta. This is considered to be one of the reasons why the protein-containing composition used in Experiment 13 was more likely to adhere to teeth, but since the oral processing time Tb was still shorter than the oral processing time Ta of the protein powder alone, it was found that it is effective to prepare a protein-containing composition by adding corn syrup. Further, it has been found that when a protein powder having a low contact angle θb is mixed with a saccharide or the like, the oral processing time may be significantly shortened as compared with the case where the protein powder is eaten alone.

### REFERENCE SIGNS LIST

10 Protein powder
11 Food
31 Saccharide
32 Protein-containing composition

1. A protein powder which has a contact angle with water of 90° or less within 60 seconds from the time point of contact with water.

2. The protein powder according to claim 1, wherein when 0.3 g of the protein powder is placed on the center of a tongue of a subject, an upper jaw of the subject is brought into contact with the tongue and allowed to stand, and after a lapse of 10 seconds from the standing, the subject is made to eat the protein powder immediately and swallow all of the protein powder, a period of time from when 10 seconds have elapsed from the standing to when the subject swallows all of the protein powder measured as the oral processing time is 70 seconds or less.

3. A protein-containing composition comprising a mixture of the protein powder according to claim 1 and a saccharide.

4. A method for producing a food, the method comprising:
a determination step of measuring a contact angle of a protein powder with water to determine whether or not a contact angle condition is satisfied on the condition that the measured contact angle of the protein powder is 90° or less within 60 seconds from the time point of contact with water; and
a food preparation step of processing the protein powder satisfying the contact angle condition to prepare a food.

5. The method for producing a food according to claim 4, wherein the determination step includes producing a protein powder for measurement from a raw material and measuring a contact angle of the protein powder for measurement; and the food preparation step includes producing a protein powder to be a food from the raw material of the protein powder for measurement determined to satisfy the contact angle condition.

6. The method for producing a food according to claim 4, wherein the determination step includes measuring a contact angle of a protein powder for measurement obtained as a part of the protein powder produced from a raw material; and the food preparation step includes processing the protein powder for which the protein powder for measurement determined to satisfy the contact angle condition has been obtained to prepare a food.

7. The method for producing a food according to claim 4, wherein the food preparation step includes mixing the protein powder with a saccharide to prepare a food.

8. A method for producing a food containing a protein powder, comprising using a protein powder having a contact angle with water of 90° or less within 60 seconds from the time point of contact with water.

9. The method for producing a food according to claim 8, wherein the food containing a protein powder is a food obtained by mixing the protein powder and a saccharide.

10. A method for producing a protein powder, the method comprising:
a determination step of producing a protein powder for measurement from a raw material and measuring a contact angle of the protein powder for measurement with water, to determine whether or not a contact angle condition is satisfied on the condition that the measured contact angle of the protein powder is 90° or less within 60 seconds from the time point of contact with water; and
a production step of producing a protein powder from the raw material of the protein powder for measurement determined to satisfy the contact angle condition.

11. A method for producing a protein-containing composition, the method comprising:
a determination step of producing a protein powder for measurement from a raw material and measuring a contact angle of the protein powder for measurement with water, to determine whether or not a contact angle condition is satisfied on the condition that the measured contact angle of the protein powder is 90° or less within 60 seconds from the time point of contact with water;
a production step of producing a protein powder from the raw material of the protein powder for measurement determined to satisfy the contact angle condition; and
a mixing step of mixing the produced protein powder with a saccharide.

12. A method for evaluating a protein powder, the method comprising:
a powder preparing step of putting a protein powder into a container and flattening the surface of the protein powder by leveling;
an adhesion step of adhering a water droplet to the surface of the protein powder;
a measurement step of measuring a contact angle of the water droplet on the surface of the protein powder; and
an evaluation step of evaluating the ease of eating the protein powder based on whether or not the contact angle is 90° or less within 60 seconds from the time point when the water droplet contacts the surface of the protein powder.

13. A method for evaluating a protein-containing composition, the method comprising:
a protein-containing composition preparing step of putting a protein-containing composition obtained by mixing a protein powder and a saccharide into a container and flattening the surface of the protein-containing composition by leveling;
an adhesion step of adhering a water droplet to the surface of the protein-containing composition;
a measurement step of measuring a contact angle of the water droplet on the surface of the protein-containing composition; and
an evaluation step of evaluating the ease of eating the protein-containing composition based on whether or not the contact angle is 90° or less within 60 seconds from the time point when the water droplet contacts the surface of the protein-containing composition.
